(19) United States
(12) Patent Application Publication
(10) Pub. No.: US 2015/0351686 A1
(43) Pub. Date: Dec. 10, 2015

(54) Electronic Watch, Personal Physiology Monitoring System and Method Thereof

(71) Applicant: GOLDTEK TECHNOLOGY CO., LTD., Taipei City (TW)

(72) Inventors: Kuang Hui Su, Taipei City (TW); Tai Chuan Chen, Taipei City (TW); Chien Lung Huang, Taipei City (TW)

(21) Appl. No.: 14/569,832

(22) Filed: Dec. 15, 2014

(30) Foreign Application Priority Data
Jun. 10, 2014 (TW) 103210152

(51) Int. Cl. A61B 5/00; A61B 5/22; A61B 5/0205; G04G 7/00; A61B 5/11

(52) U.S. Cl. CPC A61B 5/681; G04G 7/00; A61B 5/7264; A61B 5/0022; A61B 5/746; A61B 5/1112; A61B 5/02055; A61B 5/7282; A61B 5/747; A61B 5/222; A61B 2562/0223; A61B 5/02405

(57) ABSTRACT

The present invention generally relates to an electronic watch, a personal physiology monitoring system and a method thereof. The electronic watch may adapt to communicate with at least one intelligent electronic device and at least one electronic transceiver. The electronic watch comprises a first Bluetooth device, a second Bluetooth device and a display screen. The first Bluetooth device may transmit a first signal between the electronic watch and the electronic transceiver. The second Bluetooth device may transmit a second signal between the electronic watch and the intelligent electronic device. The display screen may display a first information corresponded to the first signal and a second information corresponded to the second signal.

### CROSS-REFERENCE TO RELATED APPLICATION

This application claims priority to and the benefit of Taiwan Patent Application No. 103210152 filed in the Taiwan Intellectual Property Office (TIPO) on Jun. 9, 2014, the entire contents of which are incorporated herein by reference.

### BACKGROUND OF THE INVENTION

1. Field of Invention

The present invention generally relates to an electronic watch, a personal physiology monitoring system and a method thereof, more particularly to a personal physiology monitoring system incorporating an electronic watch with two Bluetooth devices inside and a method for personal physiology monitoring.

2. Background of the Invention

For the past decades, the world is developed very fast due to gradual improved education and rapid information flow in the internet, therefore people gradually focus on the health issue because of health or sports information has become easily to access. Accordingly, the amount of persons who periodically exercise is going up year after year.

In practical exercising situation, many people prefer to aware of what the physiological results of exercise are while they are exercising; for instance, sports time period, the physiology data of heartbeat rate, calorie consumption, and running distance, etc., are concerned by those exercise lovers. Although most indoor sports devices have the function to gain the physiology data, some people still love outdoor sports. Nowadays, the smart phone is able to calculate for gaining the physiology data, but it is inconvenient for exercise, even the smart phone utilizing bridles to be attached on an arm is still inconvenient; furthermore, the smart phone is easily gotten dirt as well.

Accordingly, sporting watch with function of calculating physiology data is more favorable. However, the physiological data able to be measured is merely limited to few types, therefore needs some other more measuring devices to detect and monitor the human physiological signal. In some scenarios, the PC is utilized and installed a plurality of software (i.e. App) for outputting, converting or recording those physiological results; but it's really inconvenient. In this manner, the present physiological sensing device currently is unfavorable.

In addition, personal stuff being kept in a safe place while in exercise is also a concern, especially for outdoor sports.

Therefore, how to figure out aforesaid shortcomings in order to easily gain and monitor physiology data and protect personal stuff are important issues to persons skilled in the art.

### SUMMARY OF THE INVENTION

The main objective of the present invention is to incorporate the personal physiology monitoring system into a sporting watch, in order to detect, record and monitor the personal physiological data of an outdoor exerciser though a convenient way.

The second objective of the present invention is to measure a plurality of types of physiology data simultaneously while in exercise.

The third objective of the present invention is to prevent the scientific gadget (i.e. electronic watch) from easily stealing.

To achieve the foregoing and other objects, an electronic watch adapted to communicate with at least one intelligent electronic device and at least one electronic transceiver is disclosed. The electronic watch comprises a first Bluetooth device, a second Bluetooth device and a display screen. The first Bluetooth device may transmit a first signal between the electronic watch and the electronic transceiver. The second Bluetooth device may transmit a second signal between the electronic watch and the intelligent electronic device. The display screen may display a first information corresponded to the first signal and a second information corresponded to the second signal.

In the aforementioned electronic watch, further comprising a GPS device disposed in the electronic watch, wherein the GPS device carries out a positioning process in every single predetermined time and calculates the moving distance, moving tracks or moving speed through positioning locations and time interval of the predetermined time.

In the aforementioned electronic watch, further comprising a warning device keeping a predetermined distance away from the electronic watch or the intelligent electronic device and receiving at least one first warning signal from the electronic watch or the intelligent electronic device at a fixed frequency, and forwarding at least one second warning signal back to the electronic watch or the intelligent electronic device; wherein the electronic watch or the warning device sounds a warning voice and the intelligent electronic device transmits a notice signal to the electronic watch while the warning device is unable to receive the first warning signal, or either the electronic watch or the intelligent electronic device is unable to receive the second warning signal.

In the aforementioned electronic watch, further comprising an ANT⁺ communication device disposed in the electronic watch for providing a low power-consumption ANT⁺ communication protocol, wherein the electronic watch receives the first signal from the electronic transceiver via the ANT⁺ communication device.

To achieve the foregoing and other objects, a personal physiology monitoring system is disclosed. The personal physiology monitoring system comprises the aforesaid electronic watch, at least one electronic transceiver, at least one intelligent electronic device and a cloud platform. The cloud platform communicates with the intelligent electronic device for transmitting a third signal from the intelligent electronic device to the cloud platform, so that the cloud platform analyzes the third signal.

In the aforementioned personal physiology monitoring system, wherein the electronic watch further comprises a warning device keeping a predetermined distance away from the electronic watch or the intelligent electronic device and receiving at least one first warning signal from the electronic watch or the intelligent electronic device at a fixed frequency, and the warning device forwards at least one second warning signal back to the electronic watch or the intelligent electronic device; wherein the electronic watch or the warning device sounds a warning voice and the intelligent electronic device transmits a notice signal to the electronic watch while the warning device is unable to receive the first warning signal, or either the electronic watch or the intelligent electronic device is unable to receive the second warning signal.

In the aforementioned personal physiology monitoring system, wherein the electronic transceiver is a heartbeat sensor having two conductive members adhered to chest of a human being for measuring a tiny potential difference of heart in the human being while the heartbeat is in progress.

In the aforementioned personal physiology monitoring system, wherein the electronic transceiver is a bike cadence and speed sensor disposed adjacent to frame of the bike and having two magnets and two sensors corresponded to two magnets; wherein two magnets are respectively disposed on the pedal and wheel of a bike and two sensors detects the magnetic forces of two magnets.

In the aforementioned personal physiology monitoring system, wherein the electronic transceiver is an indoor electronic sports device for calculating the heartbeat rate of a user through an induction handle, or inputting weight and height of the user so as to calculate calorie consumption.

In the aforementioned personal physiology monitoring system, wherein the electronic watch further comprises a GPS device disposed in the electronic watch for carrying out a positioning process in every single predetermined time and calculating the moving distance, moving tracks or moving speed through positioning locations and time interval of the predetermined time.

In the aforementioned personal physiology monitoring system, wherein the electronic watch further comprises an ANT⁺ communication device disposed in the electronic watch for providing a low power-consumption ANT⁺ communication protocol, wherein the electronic watch receives the first signal from the electronic transceiver via the ANT⁺ communication device.

To achieve the foregoing and other objects, a method for personal physiology monitoring is disclosed. The method for personal physiology monitoring comprises the following steps: (A) a user wearing the aforesaid personal physiology monitoring system; (B) the electronic transceiver detecting a personal physiology signal of the user and transforming the personal physiology signal to a first signal, then the first signal being transmitted to the electronic watch via a first Bluetooth device; (C) the electronic watch receiving the first signal and transforming the first signal to a second signal, and then transmitting the second signal to the intelligent electronic device via a second Bluetooth device; (D) the intelligent electronic device transforming the second signal to a third signal, and then transmitting the third signal to the cloud platform for analysis; and (E) if the result of the analysis is abnormal, the cloud platform notifying a server of an ambulance department.

In the aforementioned method for personal physiology monitoring, wherein the analyzed result of step (E) is abnormal if one of the following conditions is positive: (1) the heartbeat exceeding 150 beats/min.; (2) the body temperature lower than 35° C.; (3) the body temperature higher than 39° C.; (4) the blood sugar lower than 50 mg/dL.

In the aforementioned method for personal physiology monitoring, wherein the electronic watch further comprises an ANT⁺ communication device, and the electronic watch receives the first signal via the ANT⁺ communication device in step (B). To further understand the techniques, means and effects of the instant disclosure applied for achieving the prescribed objectives, the following detailed descriptions and appended drawings are hereby referred, such that, through which, the purposes, features and aspects of the instant disclosure can be thoroughly and concretely appreciated. However, the appended drawings are provided solely for reference and illustration, without any intention to limit the instant disclosure.

### BRIEF DESCRIPTION OF THE DRAWINGS

The objects, spirits, and advantages of the preferred embodiments of the present invention will be readily understood by the accompanying drawings and detailed descriptions, wherein:

FIG. 1A illustrates a block diagram of a personal physiology monitoring system according to the present invention;

FIG. 1B illustrates a schematic view of an electronic watch according to the present invention;

FIG. 2A illustrates a schematic view of the electronic watch communicating with the electronic transceiver according to the present invention;

FIG. 2B illustrates a schematic view of the electronic watch communicating with the electronic transceiver via an ANT⁺ communication device according to the present invention;

FIG. 2C is a calorie consumption table of sports;

FIG. 3A and FIG. 3B illustrate a schematic view of the electronic watch communicating with an intelligent electronic device according to the present invention;

FIG. 4 illustrates a relationship between a warning device, the electronic watch and the intelligent electronic device according to the present invention;

FIG. 5 illustrates an analyzing table produced by the cloud platform according to the present invention; and

FIG. 6 is a flow chart of for monitoring personal physiological data according to the present invention.

### DESCRIPTION OF EMBODIMENTS

Following preferred embodiments and figures will be described in detail so as to achieve aforesaid objects.

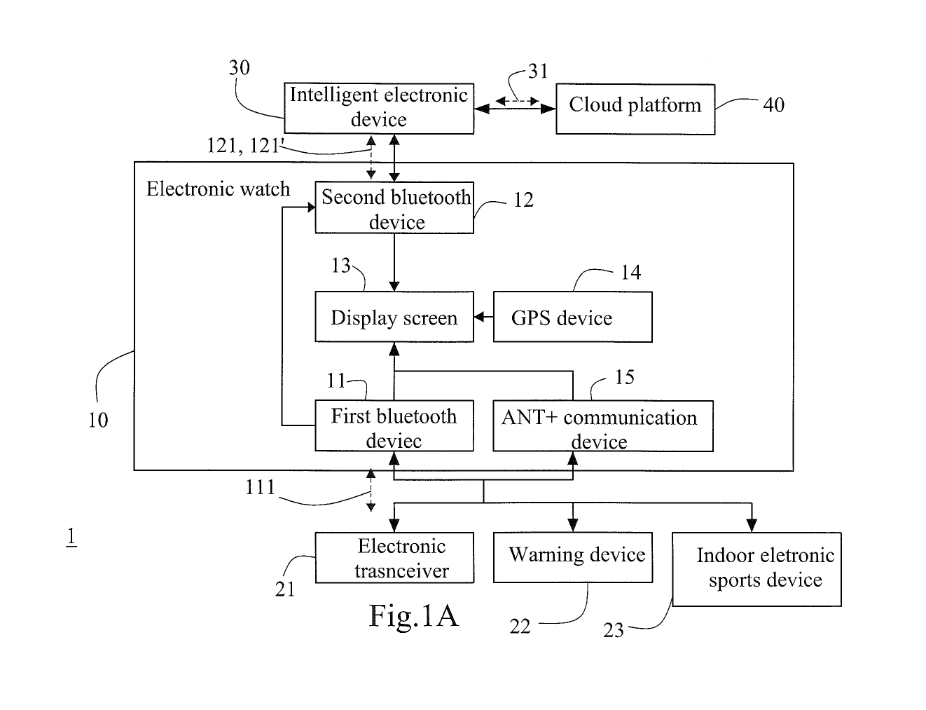

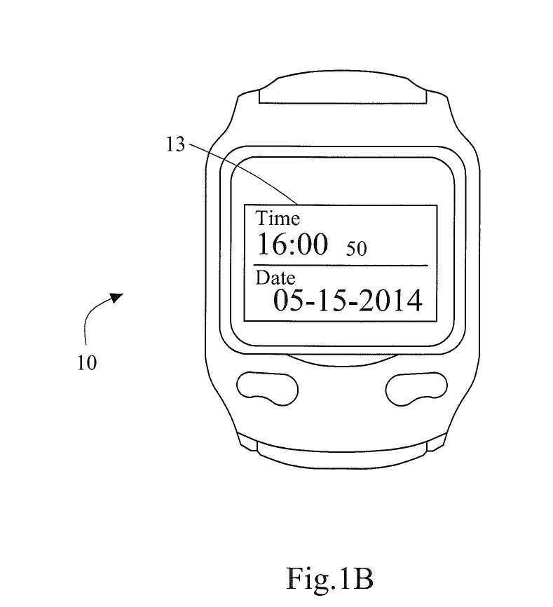

Please refer to FIG. 1A and FIG. 1B, which illustrate a block diagram of a personal physiology monitoring system and a schematic view of an electronic watch according to the present invention. A personal physiology monitoring system 1 comprises an electronic watch 10, an electronic transceiver 21, a warning device 22, and an intelligent electronic device 30. The electronic watch 10 has a first Bluetooth device 11, a second Bluetooth device 12 and a display screen 13. The electronic watch 10 communicates with the electronic transceiver 21 via the first Bluetooth device 11, and the electronic watch 10 and the electronic transceiver 21 transmits a first signal 111 to each other; i.e. the first Bluetooth device 11 may transmit the first signal 111 between the electronic watch 10 and the electronic transceiver 21. Preferably, the electronic transceiver 21 can be a physiological sensor such as heartbeat sensor, pedometer, calorie consumption analyzer, bike cadence and speed sensor, etc. The electronic watch 10 communicates with the intelligent electronic device 30 via the second Bluetooth device 12, and the electronic watch 10 and the intelligent electronic device 30 transmit a second signal 121 to each other; as a result, the second Bluetooth device 12 may transmit a second signal 121, 121′ between the electronic watch 10 and the intelligent electronic device 30. The intelligent electronic device 30 can be a tablet PC or a smart phone.

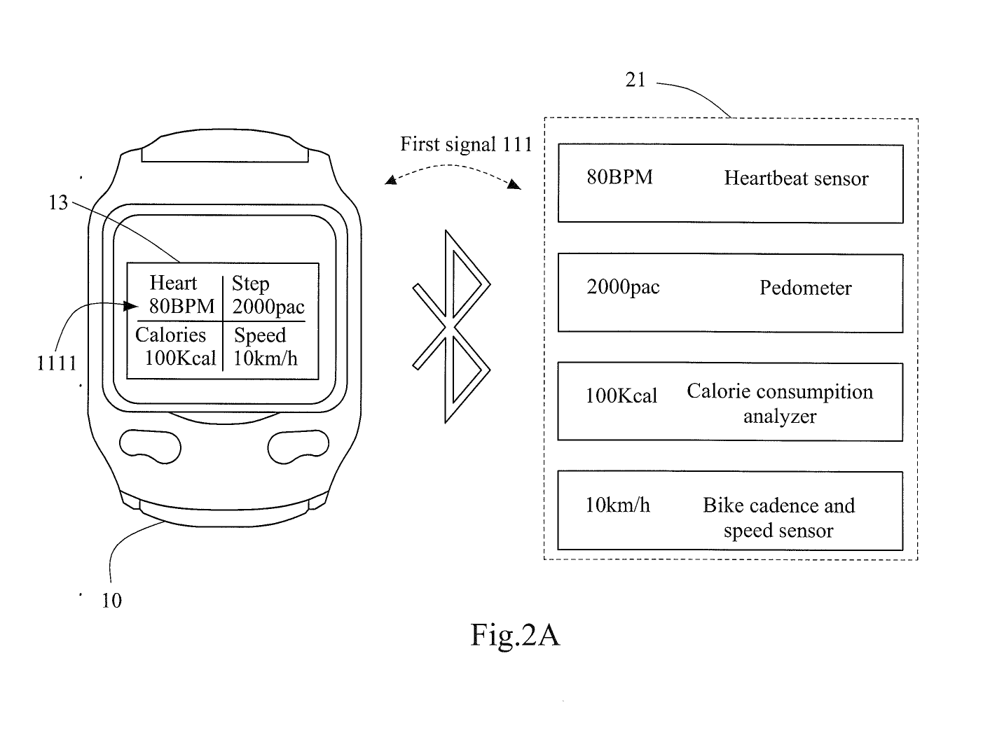

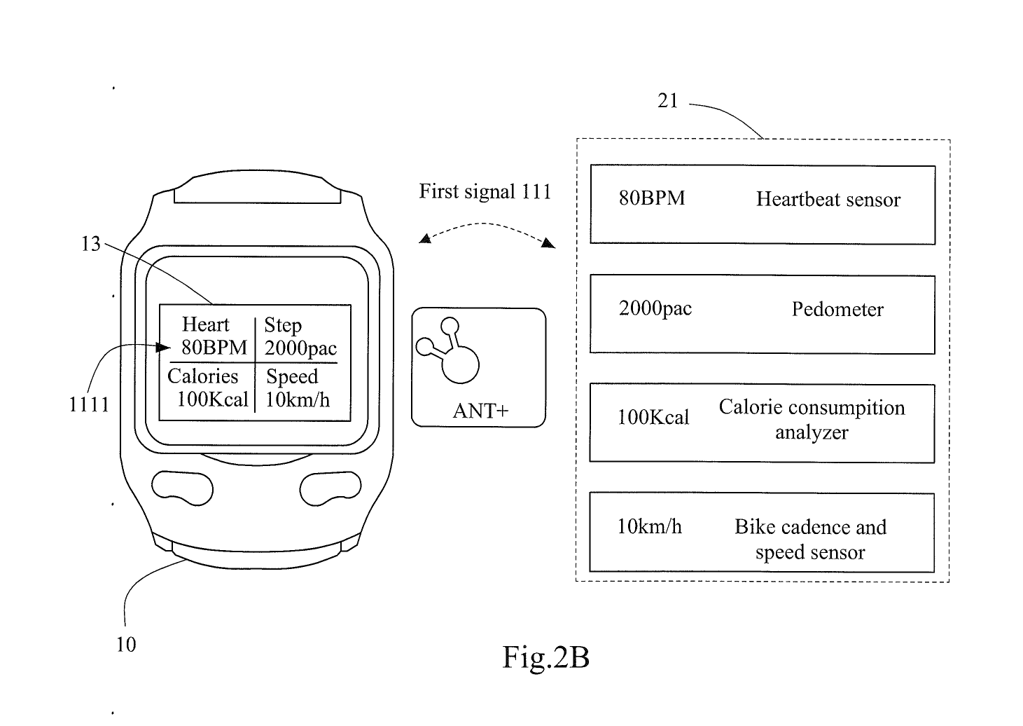

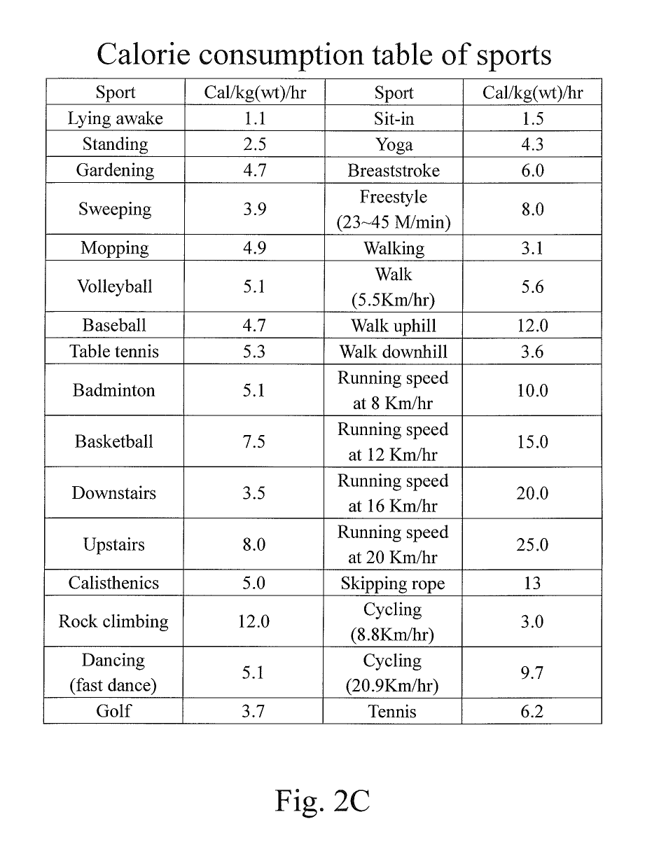

Referring to FIG. 2A, which illustrates a schematic view of the electronic watch communicating with the electronic transceiver according to the present invention. The electronic transceiver 21 is able to measure the physiological signal of a user who have the electronic watch 10 on his/her arm, in which the physiological signal can be heart rate or calories of the user, or amount of steps or bicycle speed. Afterward the physiological signal is transformed into a first signal 111, and then the electronic watch 10 receives the first signal 111 through the first Bluetooth device 11. Hence, the electronic watch 10 may display a first electronic information 1111 corresponding to the first signal 111 on the display screen 13.

The heartbeat sensor has two conductive members. The two conductive members are adhered to the chest of a human being so as to measure the tiny potential difference of heart in the human being while the heartbeat is in progress. In this manner, a present heartbeat rate or frequency can be achieved, and afterward transformed into the first signal 111 and sent to the electronic watch 10. The bike cadence and speed sensor has two sensors. Two magnets corresponding to these two sensors are disposed on the pedal and wheel of a bike respectively; the bike cadence and speed sensor adjacent to the pedal and the wheel are disposed on the frame of the bike. When the pedal and the wheel start rotation, the two magnets may pass these two corresponding sensors, therefore the bike cadence and speed sensor is able to measure the stepping frequency of the present bike and the rotation amount, rotation rate or speed of the bike is hence achieved. Afterward, the stepping frequency and the bike speed are transformed into the first signal 111, and then the first signal 111 is transmitted to the electronic watch 10.

The electronic watch 10 may also communicates with an indoor electronic sports device 23 via the first Bluetooth device 11. The indoor electronic sports device 23 is also able to measure the physiological information of an exerciser, or calculates the heartbeat rate or frequency of an exerciser through an induction handle, or input the weight/height of the exerciser so as to analyze calorie consumption in an exercising time period.

Please refer to FIG. 2C, which is a calorie consumption table of sports. The electronic watch 10 may have the calorie consumption table stored, and the exerciser may input data, such as height or weight of the exerciser, into the electronic watch 10 or choose a specific sport. When exercising, the electronic watch 10 is capable of calculating calorie consumption by means of the weight of the exerciser and the calorie consumption table. When the electronic watch 10 detects the location of the exerciser is not changed via a GPS device 14, the electronic watch 10 may automatically stop calculation of the calorie consumption. If the exerciser is operating the indoor electronic sports device 23, the calorie consumption calculation may be automatically shut down. Practically, the table shown in FIG. 2C is an example; and people skilled in the art may also have some other types of sports to be utilized in the electronic watch 10 according to the present invention.

In addition, referring to FIG. 2B, which illustrates a schematic view of the electronic watch communicating with the electronic transceiver via an ANT⁺ communication device according to the present invention. The electronic watch 10 may further have an ANT⁺ communication device 15 disposed inside, in which the ANT⁺ is a low power-consumption communication protocol and is broadly applied in the field of medical, especially in transmitting physiological signals. Therefore, the electronic transceiver 21 may adopt the ANT⁺ communication protocol for transmission. Consequently, the electronic watch 10 is able to receive the first signal 111 by means of the electronic transceiver 21 and the ANT⁺ communication device 15, and then display the first electronic information 1111 corresponding to the first signal 111 on the display screen 13.

The electronic watch 10 further has a GPS device 14 disposed inside, wherein the GPS device 14 carries out a positioning process in every single predetermined time and records the time and location; moreover, it may calculate the moving distance, moving tracks or moving speed of a positioning time interval by means of the recorded positioning time point and the positioning site. Besides, the moving distance and moving speed may also be shown on the display screen 13 as well.

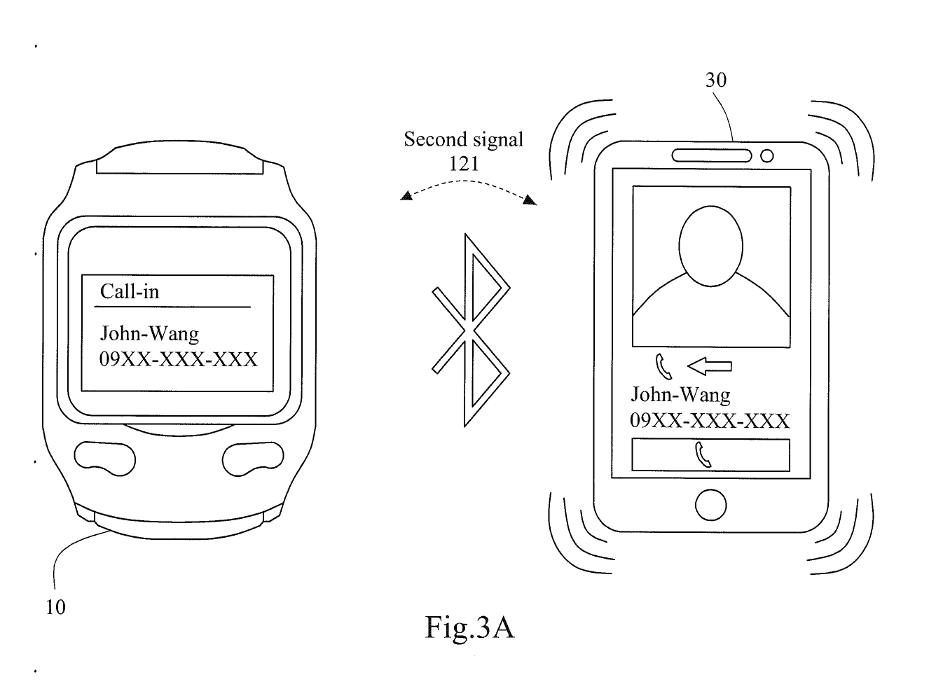

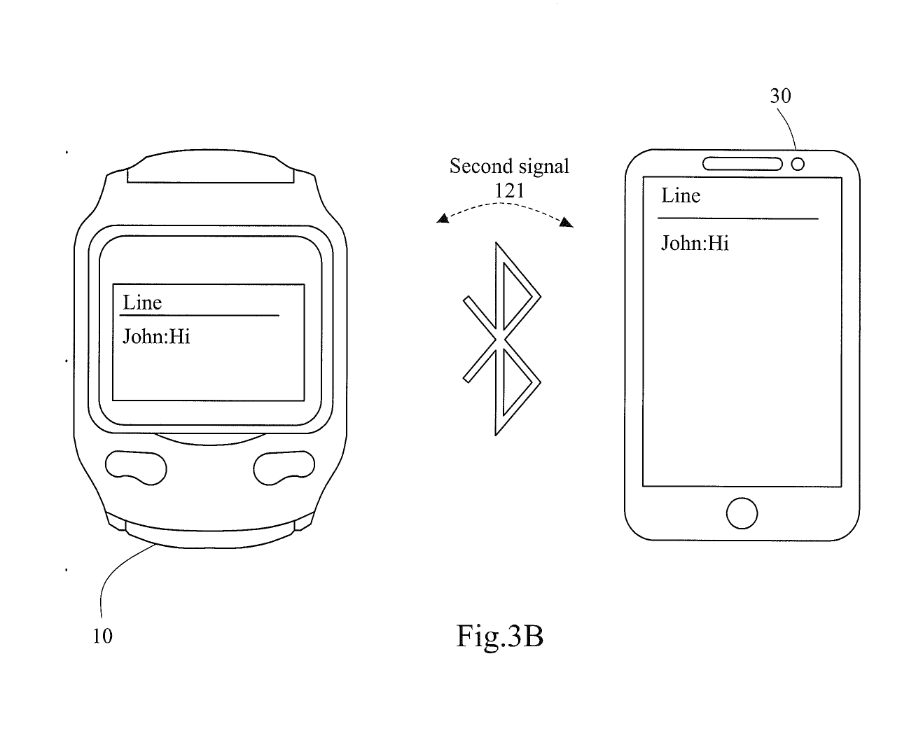

Please refer to FIG. 3A and FIG. 3B, which illustrate a schematic view of the electronic watch communicating with an intelligent electronic device according to the present invention. As shown in FIG. 3A, when a call is made to the intelligent electronic device 30, the intelligent electronic device 30 may transform the information, such as number of the call or name of the dialer, into a second signal 121 and then send the second signal 121 to the electronic watch 10. The electronic watch 10 receives the second signal 121 via the second Bluetooth device 12, then the second electronic information corresponded to the second signal 121 is able to be revealed on the display screen 13. As shown in FIG. 3B, when the intelligent electronic device 30 receives the notice of a communication software or App, such as Line, Facebook, Skype, Twitter, etc., the intelligent electronic device 30 may transform the notice into the second signal 121, and afterward the second signal 121 is transmitted to the electronic watch 10. Furthermore, the electronic watch 10 may show the notice of the communication software or App on the display screen 13. Practically, the aforesaid notice may be text information (e.g. the name, number or digital condition of communication software, etc.) or other graphical image (e.g. emotional image); similarly, the graphical image can also be displayed on the display screen 13.

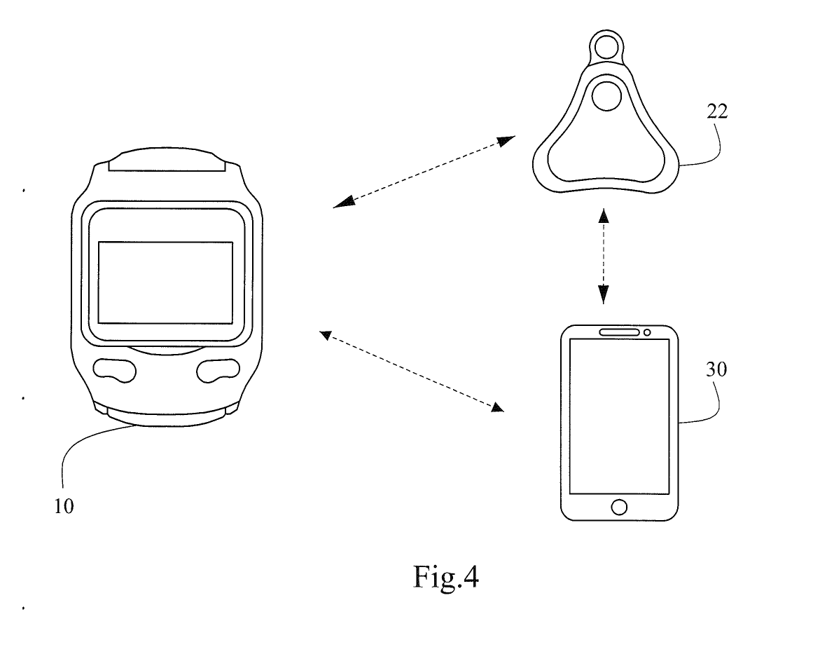

Please refer to FIG. 4, which illustrates a relationship between a warning device, the electronic watch and the intelligent electronic device according to the present invention. The warning device 22 is external to the electronic watch 10, that is, the warning device 22 is a predetermined distance away from the electronic watch 10. The warning device 22 may receive a first warning signal from the electronic watch 10 or the intelligent electronic device 30 through an invariant frequency and forwards a second warning signal back to the electronic watch 10 or the intelligent electronic device 30. When the warning device 22 is not able to receive the first warning signal, or the electronic watch 10 or the intelligent electronic device 30 is not able to receive the second warning signal, i.e. the warning device 22 and the electronic watch 10 (or the intelligent electronic device 30) is far away rather than the predetermined distance, the electronic watch 10 or the intelligent electronic device 30 may sound a warning voice (or vibrate) to notify the owner. In this manner, the warning device 22 can be carried on a cellular phone, purse or backpack, or a pet, child, elderly, etc., that requires special attentions. While going out, the electronic watch 10 or the intelligent electronic device 30 may sound a voice (or vibrate) to notify the owner for paying more attention to the personal stuff or their relatives if the warning device 22 is too far away from the electronic watch 10 or the intelligent electronic device 30 (far away rather than the predetermined distance).

Please refer to FIG. 1A again, the personal physiology monitoring system 1 may further comprise a cloud platform 40. The electronic watch 10 may receive physiological signals from the electronic transceiver 21 or the indoor electronic sports device 23, and transmits the physiological signals to the cloud platform 40. The physiological signal measured or detected by the electronic transceiver 21 or the indoor electronic sports device 23 may be transformed into the first signal 111, and the first signal 111 may be transmitted to the first Bluetooth device 11 of the electronic watch 10. The electronic watch 10 can transform the first signal 111 into the second signal 121′, and the second signal 121′ is able to be delivered to the intelligent electronic device 30 by means of the second Bluetooth device 12. Afterward, the intelligent electronic device 30 may transform the second signal 121′ into the third signal 31, and then the third signal 31 is able to be transmitted to the cloud platform 40. In this manner, the cloud platform 40 may stores the physiology signal of the third signal 31 or other sports information, so as to analyze the physiology signal and sports information.

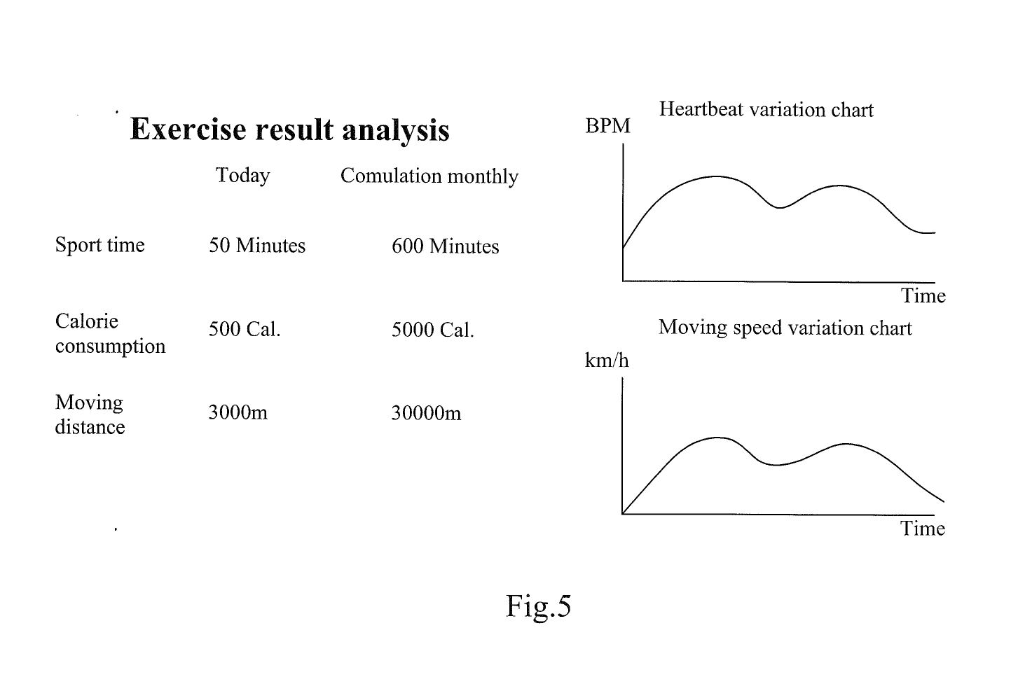

With reference to FIG. 5, which illustrates an analyzing table produced by the cloud platform according to the present invention. After the cloud platform 40 receives and analyzes the third signal 31, the analyzed exercise result is therefore achieved according to the third signal 31. This analyzing table represent the contents of the third signal 31 within a certain time period. The certain time period can be a month, two weeks, one week, etc., and it is changed according to the user. The types of the contents analyzed may be calorie consumption statistics, heartbeat rate lines, exercise time line statistics, etc. The user can monitors or downloads the analyzed exercise results through the intelligent electronic device 30 or other electronic devices connecting with the Internet. Further, user may change his own sports habit according to the analyzed exercise results.

In addition to the physiological signal or sports information measured by the electronic transceiver 21 or the indoor electronic sports device 23, the values, such as the geographic data of the moving distance, moving tracks or moving speed, etc., calculated by the GPS device 14 are transformed into the second signal 121′, and the second signal 121′ is transmitted to the intelligent electronic device 30. Thereafter the intelligent electronic device 30 transforms the second signal 121′ into the third signal 31, and then the third signal 31 is transmitted to the cloud platform 40. The cloud platform 40 is therefore able to analyze the geographic data in order to achieve an analyzing table or chart such as moving speed variation chart, moving tracks in a map, or actual moving time, etc.

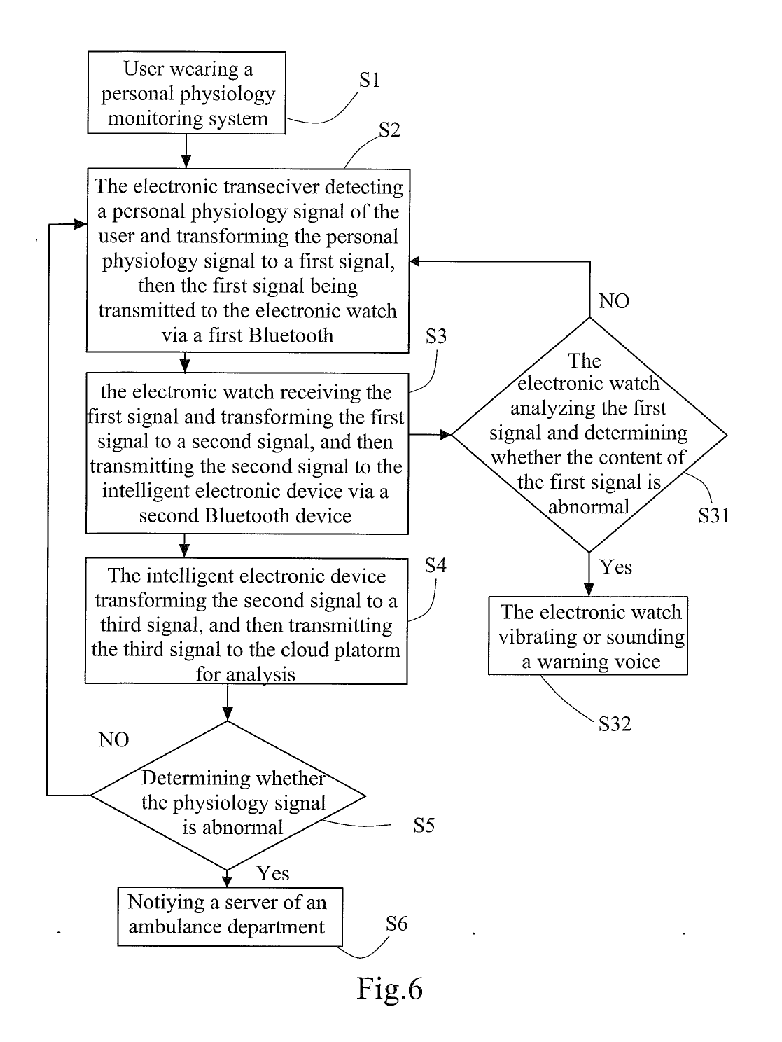

With reference to FIG. 6, which is a flow chart of for monitoring personal physiological data according to the present invention. The method comprises the steps of: (Step S1) user wearing a personal physiology monitoring system 1; (Step S2) the electronic transceiver 21 detecting a personal physiological signal of the user and transforming the personal physiology signal to a first signal 111, then the first signal 111 being transmitted to the electronic watch 10 via a first Bluetooth device 11; (Step S3) the electronic watch 10 receiving the first signal 111 via the first Bluetooth device 11, and analyzing and recording the first signal 111, then the electronic watch 10 transforming the first signal 111 to a second signal 121′, and the second signal 121′ being transmitted to the intelligent electronic device 30 via a second Bluetooth device 12; (Step S31) the electronic watch 10 analyzing the first signal 111 and determining whether the contents of the first signal 111 is abnormal; (Step S32) the electronic watch 10 vibrating or sounding a warning voice (or vibrate) to notify the user; (Step S4) the intelligent electronic device 30 transforming the second signal 121′ into the third signal 31 and the third signal 31 being transmitted to the cloud platform 40 after the intelligent electronic device 30 receives the second signal 121′, wherein the cloud platform 40 analyzing the third signal 31 so as to achieve the analyzed result; (Step S5) determining whether the physiology signal is abnormal; and (Step S6) the cloud platform 40 notifying a service of an ambulance department if the judgment of Step S5 is yes.

In Step S31, the regarded abnormal conditions determined by the electronic watch 10 analyzing the first signal 111 can be one of the following: the heartbeat exceeding 150 beats/min, the body temperature being lower than 35° C., the body temperature being higher than 39° C., and the blood sugar being lower than 50 mg/dL. The aforementioned conditions are commonly seen, and people skilled in the art may have more conditions applied according to user's health or conditions. Besides, the physiology data such as heartbeat, body temperature, blood sugar, etc. can be analyzed by the electronic watch 10; however, more complicated data such as moving distance, calorie consumption, heartbeat variety, etc. are analyzed by the cloud platform 40.

Additionally, after the intelligent electronic device 30 receives the second signal 121′, the user is able to directly monitor the contents of the second signal 121′, hence the user may instantly check the physiological signals himself through the electronic watch 10 and the intelligent electronic device 30. After the third signal 31 is transmitted to the cloud platform 40, the cloud platform 40 may either analyze the third signal 31 or publish to the web or a digit media, so as to share and discuss his exercising results with a social community. Further, the cloud platform 40 may communicate with medical service units (e.g. hospital, clinics or emergency medical team). After the third signal 31 is transmitted to the cloud platform 40, the cloud platform 40 may afterward transmit the physiological information (e.g. physician of the user) reside in the third signal 31 to the medical service units. In this manner, the medical service units are able to monitor the physiology data of the user instantly, and provides professional suggestions to the user based on those physiology data.

Compared to prior sports watch or other wearable devices for physiology monitoring, the personal physiology monitoring system 1 or electronic watch 10 according to the present invention can monitor the physiology signals of a user instantly, so as to evaluate one's health conditions or check one's exercising results. Furthermore, several types of physiology signals can be integrated as well, so that only one software (or App) is capable of processing the data captured from different devices or systems. Besides, the electronic watch 10 is equipped with or adapted to several warning devices 22, and therefore the user is capable of effectively managing personal stuff for avoiding steal.

Alternatively, the personal physiology monitoring system 1 is able to be applied to the medical care system, therefore some medical service units may keep track of patient's status or conditions by means of the cloud platform 40 anytime. If the physiology data of a user are abnormal, the personal physiology monitoring system 1 is able to inform or notify the medical service units instantly via the cloud platform 40, so that the medical service units can process in the first stage with very high efficiency.

The above-mentioned descriptions merely represent the preferred embodiments of the instant disclosure, without any intention or ability to limit the scope of the instant disclosure which is fully described only within the following claims. Various equivalent changes, alterations or modifications based on the claims of instant disclosure are all, consequently, viewed as being embraced by the scope of the instant disclosure.

What is claimed is:

1. An electronic watch (10) adapted to communicate with at least one intelligent electronic device (30) and at least one electronic transceiver (21), comprising:
a first Bluetooth device (11) for transmitting a first signal (111) between the electronic watch (10) and the electronic transceiver (21);
a second Bluetooth device (12) for transmitting a second signal (121, 121′) between the electronic watch (10) and the intelligent electronic device (30); and
a display screen (13), displaying a first information corresponded to the first signal (111) and a second information corresponded to the second signal (121, 121′).

2. The electronic watch (10) in the claim 1, further comprising a GPS device (14) disposed in the electronic watch (10), wherein the GPS device (14) carries out a positioning process in every single predetermined time and calculates the moving distance, moving tracks or moving speed through positioning locations and time interval of the predetermined time.

3. The electronic watch (10) in the claim 1, further comprising a warning device (22) keeping a predetermined distance away from the electronic watch (10) or the intelligent electronic device (30) and receiving at least one first warning signal from the electronic watch (10) or the intelligent electronic device (30) at a fixed frequency, and forwarding at least one second warning signal back to the electronic watch (10) or the intelligent electronic device (30); wherein the electronic watch (10) or the warning device (22) sounds a warning voice and the intelligent electronic device (30) transmits a notice signal to the electronic watch (10) while the warning device (22) is unable to receive the first warning signal, or either the electronic watch (10) or the intelligent electronic device (30) is unable to receive the second warning signal.

4. The electronic watch (10) in the claim 1, further comprising an ANT⁺ communication device (15) disposed in the electronic watch (10) for providing a low power-consumption ANT⁺ communication protocol, wherein the electronic watch (10) receives the first signal (111) from the electronic transceiver (21) via the ANT⁺ communication device (15).

5. A personal physiology monitoring system (1), comprising:
an electronic watch (10) as claimed in the claim 1;
at least one electronic transceiver (21);
at least one intelligent electronic device (30); and
a cloud platform (40), communicating with the intelligent electronic device (30) for transmitting a third signal (31) from the intelligent electronic device (30) to the cloud platform (40), so that the cloud platform (40) analyzes the third signal (31).

6. The personal physiology monitoring system (1) in the claim 5, wherein the electronic watch (10) further comprises a warning device (22) keeping a predetermined distance away from the electronic watch (10) or the intelligent electronic device (30) and receiving at least one first warning signal from the electronic watch (10) or the intelligent electronic device (30) at a fixed frequency, and the warning device (22) forwards at least one second warning signal back to the electronic watch (10) or the intelligent electronic device (30); wherein the electronic watch (10) or the warning device (22) sounds a warning voice and the intelligent electronic device (30) transmits a notice signal to the electronic watch (10) while the warning device (22) is unable to receive the first warning signal, or either the electronic watch (10) or the intelligent electronic device (30) is unable to receive the second warning signal.

7. The personal physiology monitoring system (1) in the claim 5, wherein the electronic transceiver (21) is a heartbeat sensor having two conductive members adhered to chest of a human being for measuring a tiny potential difference of heart in the human being while the heartbeat is in progress.

8. The personal physiology monitoring system (1) in the claim 5, wherein the electronic transceiver (21) is a bike cadence and speed sensor disposed adjacent to frame of the bike and having two magnets and two sensors corresponded to two magnets; wherein two magnets are respectively disposed on the pedal and wheel of a bike and two sensors detects the magnetic forces of two magnets.

9. The personal physiology monitoring system (1) in the claim 5, wherein the electronic transceiver (21) is an indoor electronic sports device for calculating the heartbeat rate of a user through an induction handle, or inputting weight and height of the user so as to calculate calorie consumption.

10. The personal physiology monitoring system (1) in the claim 5, wherein the electronic watch (10) further comprises a GPS device (14) disposed in the electronic watch (10) for carrying out a positioning process in every single predetermined time and calculating the moving distance, moving tracks or moving speed through positioning locations and time interval of the predetermined time.

11. The personal physiology monitoring system (1) in the claim 5, wherein the electronic watch (10) further comprises an ANT⁺ communication device (15) disposed in the electronic watch (10) for providing a low power-consumption ANT⁺ communication protocol, wherein the electronic watch (10) receives the first signal (111) from the electronic transceiver (21) via the ANT⁺ communication device (15).

12. A method for personal physiology monitoring, comprising the steps of:
(A) a user wearing the personal physiology monitoring system (1) as claimed in the claim 5;
(B) the electronic transceiver (21) detecting a personal physiology signal of the user and transforming the personal physiology signal to a first signal (111), then the first signal (111) being transmitted to the electronic watch (10) via a first Bluetooth device (11);
(C) the electronic watch (10) receiving the first signal (111) and transforming the first signal (111) to a second signal (121, 121′), and then transmitting the second signal (121, 121′) to the intelligent electronic device (30) via a second Bluetooth device (12);
(D) the intelligent electronic device (30) transforming the second signal (121, 121′) to a third signal (31), and then transmitting the third signal (31) to the cloud platform (40) for analysis; and
(E) if the result of the analysis is abnormal, the cloud platform (40) notifying a server of an ambulance department.

13. The method for personal physiology monitoring in the claim 12, wherein the analyzed result of step (E) is abnormal if one of the following conditions is positive:
i. the heartbeat exceeding 150 beats/min.;
ii. the body temperature lower than 35° C.;
iii. the body temperature higher than 39° C.;
iv. the blood sugar lower than 50 mg/dL.

14. The method for personal physiology monitoring in the claim 12, wherein the electronic watch (10) further comprises an ANT⁺ communication device (15), and the electronic watch (10) receives the first signal (111) via the ANT⁺ communication device (15) in step (B).
